(19) United States
(12) Patent Application Publication
(10) Pub. No.: US 2022/0354732 A1
(43) Pub. Date: Nov. 10, 2022

(54) Rejuvenating Beauty Care Tool for Skin and Body

(71) Applicant: Guy Levi, Dania, FL (US)

(72) Inventor: Guy Levi, Dania, FL (US)

(21) Appl. No.: 17/361,115

(22) Filed: Jun. 28, 2021

Related U.S. Application Data
(63) Continuation-in-part of application No. 29/782,169, filed on May 4, 2021.

(51) Int. Cl. A61H 15/02; A61N 1/36; A61N 1/40; A61N 5/06; A61F 7/00; A61H 23/02; A61H 15/00

(52) U.S. Cl. CPC A61H 15/02; A61N 1/36003; A61N 1/403; A61N 5/0625; A61F 7/007; A61H 23/0245; A61H 15/0092; A61H 2015/0021; A61H 2201/105; A61H 2015/005; A61H 2201/1676; A61H 2201/10; A61H 2201/5007; A61H 2015/0071; A61H 2201/0285; A61H 2205/062; A61N 2005/0652; A61N 2005/0659; A61F 2007/003; A61F 2007/0075

(57) ABSTRACT

A rejuvenating beauty care tool for skin and body is an apparatus that enables the user to treat different skin and body areas using different therapeutic and cosmetic techniques. The apparatus may include a handle, a first arm, a second arm, a plurality of first rollers, and a plurality of second rollers. The handle enables the user to comfortably operate the apparatus using only one hand. The handle also supports the first arm and the second arm when the first arm and the second arm are pressed against the user's body. The first arm presses the plurality of first rollers against a first target body area. Likewise, the second arm presses the plurality of second rollers against a second target body area. Further, both the plurality of first rollers and the plurality of second rollers enable the application of the desired cosmetic or therapeutic treatment onto the target body areas.

The current application is a continuation-in-part (CIP) application of the U.S. design application Ser. No. 29/782,169 filed on May 4, 2021.

### FIELD OF THE INVENTION

The present invention relates generally to beauty care tools and therapeutic systems. More specifically, the present invention provides a skin care and therapeutic tool that heals and rejuvenates the skin and body.

### BACKGROUND OF THE INVENTION

Multiple systems and devices are currently available that help people treat their skin and body for therapeutic purposes. Likewise, various skin care and beauty products are available to help people maintain a youthful and healthy body. However, due to the large number and types of products available, it can be difficult to find one that can provide multiple cosmetic and therapeutic benefits in a single device. People often must utilize multiple products to achieve the desired results, which can be expensive for some. In addition, the process of finding the right product can often be frustrating for some. There is a need for a device that provides multiple therapeutic and cosmetic benefits in a single, portable device.

An objective of the present invention is to provide a rejuvenating beauty care tool for skin and body for anti-aging, muscle toning, skin tightening, etc. The present invention includes multiple rejuvenating and therapeutic features designed to help people to take care of their skin and body. Another objective of the present invention is to provide a rejuvenating beauty care tool for skin and body that is portable, does not require to be plugged in to operate, and can be easily operated single using one hand. Additional features and benefits of the present invention are further discussed in the sections below.

### SUMMARY OF THE INVENTION

The present invention is a rejuvenating beauty care tool for skin and body that helps users to take care of their skin and body by providing different cosmetic and therapeutic treatments such as Electrical Muscle Stimulation (EMS), Radio Frequency (RF) therapy, Light Emitting Diode (LED) therapy, vibration (sonic, ultrasonic, etc.) therapy, hot and cold therapy, etc. All these treatments are integrated into a portable, versatile device that can be operated with a single hand. In addition, the present invention can be applied onto different areas around the body to treat different skin areas or muscle groups. Thus, the user can heal and rejuvenate their skin and body without the need to utilize different tools and products.

### BRIEF DESCRIPTION OF THE DRAWINGS

FIG. 1 is a top perspective view showing the present invention.

FIG. 2 is a bottom perspective view showing the present invention.

FIG. 3 is a top perspective view showing the present invention, wherein the first arm is shown separated from the second arm.

FIG. 4 is a side view showing the present invention.

FIG. 5 is a side view showing the present invention, wherein the first arm is shown separated from the second arm.

FIG. 6 is a cross-sectional view taken along line 6-6 in FIG. 4.

FIG. 7 is a top view showing the present invention, wherein the first arm is shown separated from the second arm.

FIG. 8 is a schematic view showing the geared continuous hinge and the spring-loaded mechanism of the present invention.

FIG. 9 is a schematic view showing the electronic connections and the electrical connections of the present invention, wherein the electronic connections are shown as dashed path lines, and wherein the electrical connections are shown as solid path lines.

FIG. 10 is a schematic view showing the piezoelectric device and the thermoelectric device of the present invention, wherein the electronic connections are shown as dashed path lines, and wherein the electrical connections are shown as solid path lines, and wherein the vibrational communication is shown as dot-dash path lines, and wherein the thermal communication is shown as dash-gap path lines.

FIG. 11 is a magnified view taken from circle 11 in FIG. 5.

### DETAIL DESCRIPTIONS OF THE INVENTION

All illustrations of the drawings are for the purpose of describing selected versions of the present invention and are not intended to limit the scope of the present invention.

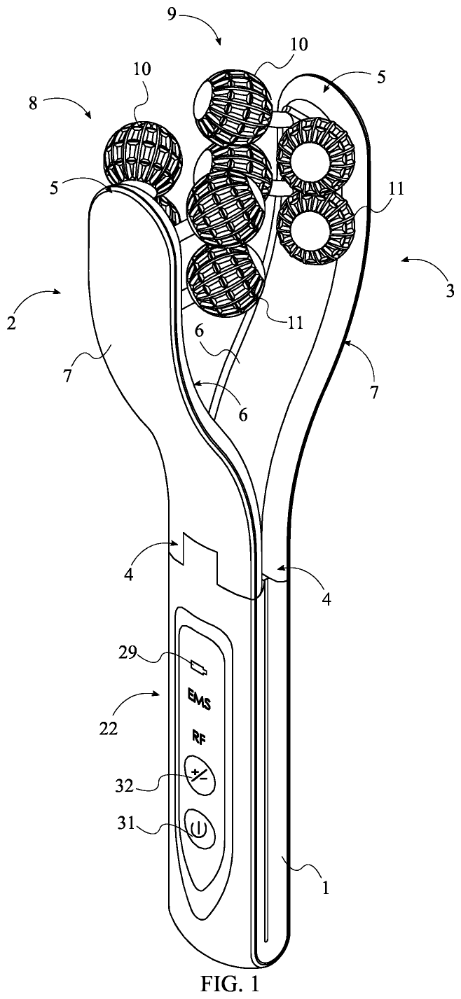

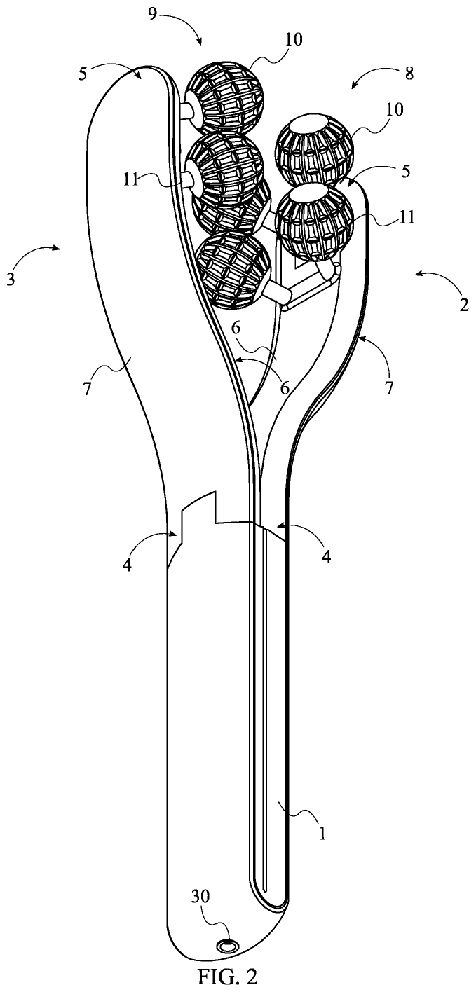

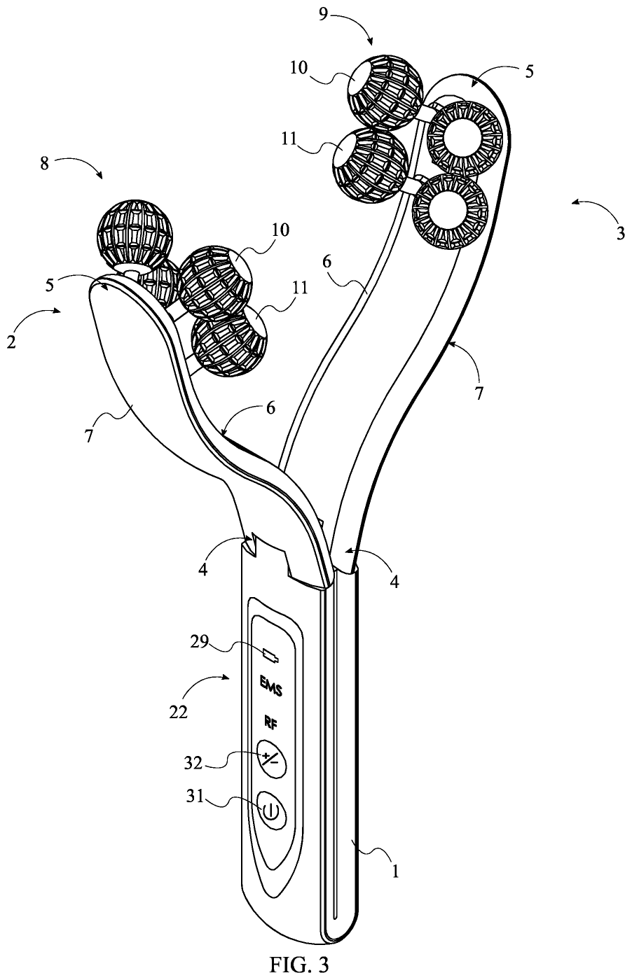

The present invention is a rejuvenating beauty care tool for skin and body that enables the user to treat different skin and body areas using different therapeutic and cosmetic techniques. As can be seen in FIG. 1 through 3, the present invention may comprise a handle 1, a first arm 2, a second arm 3, a plurality of first rollers 8, and a plurality of second rollers 9. The handle 1 enables the user to comfortably operate the present invention using only one hand. The handle 1 also supports the first arm 2 and the second arm 3 when the first arm 2 and the second arm 3 are pressed against the user's body. The first arm 2 presses the plurality of first rollers 8 against a first target body area. Likewise, the second arm 3 presses the plurality of second rollers 9 against a second target body area. Further, both the plurality of first rollers 8 and the plurality of second rollers 9 enable the application of the desired cosmetic or therapeutic treatment onto the target body areas.

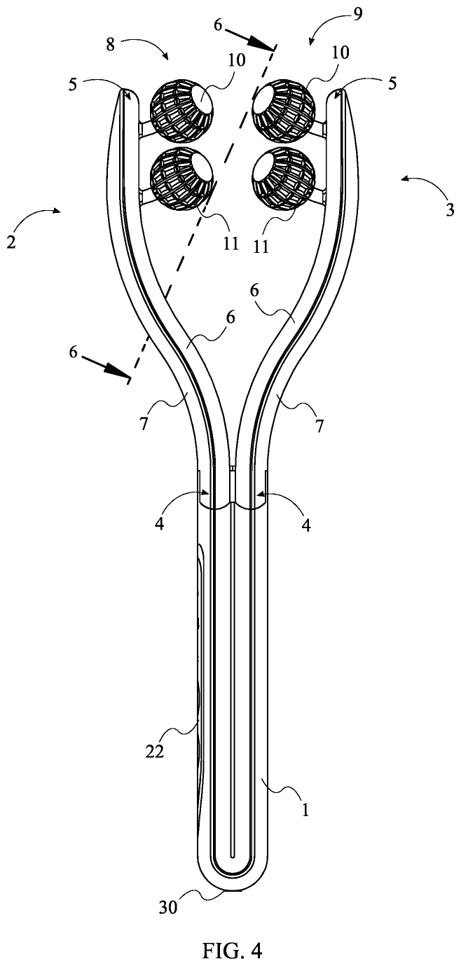

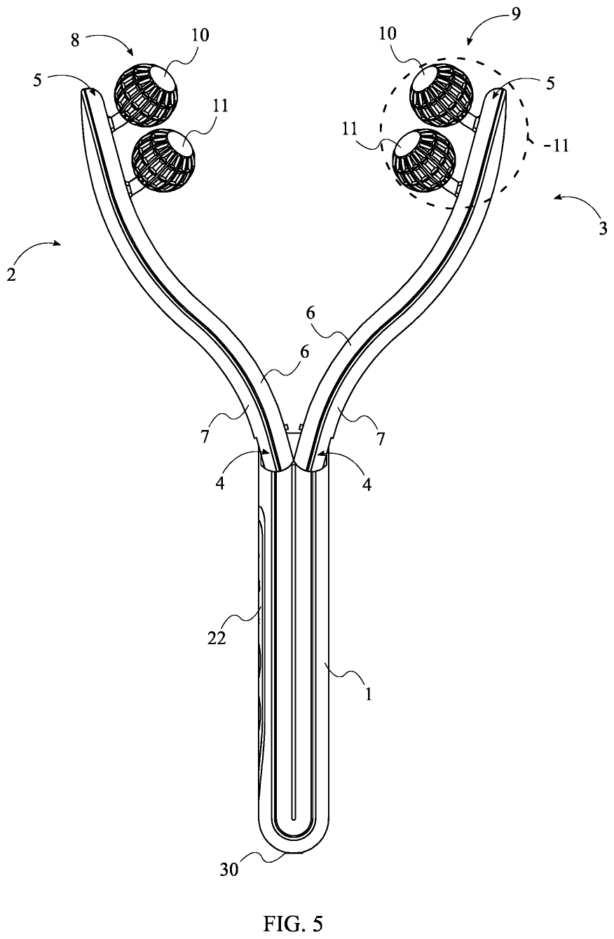

The general configuration of the aforementioned components enables users to easily target different muscles and skin areas to apply a desired treatment for healing and rejuvenation. The handle 1 is preferably an elongated thin structure that can be comfortably handled using a single hand to press the first arm 2 and the second arm 3 against the target body area. Similarly, the first arm 2 and the second arm 3 are elongated structures designed to conform to the different features of the user's body. As can be seen in FIGS. 4 and 5, the first arm 2 and the second arm 3 each comprise a proximal arm end 4, a distal arm end 5, an inner arm face 6, and an outer arm face 7. The proximal arm end 4 and the distal arm end 5 preferably correspond to the opposing ends of the corresponding arm. The inner arm face 6 and the outer arm face 7 preferably correspond to the opposing lateral surfaces of the corresponding arm. The proximal arm end 4 is hingedly and terminally connected to the handle 1 so that the corresponding arm can pivot about the proximal arm end 4. The inner arm face 6 of the first arm 2 and the inner arm face 6 of the second arm 3 are oriented towards each other. Also, the outer arm face 7 of the first arm 2 and the outer arm face 7 of the second arm 3 are oriented away from each other. Thus, the first arm 2 and the second arm 3 mirror each other. The positioning of the first arm 2 and the second arm 3 is similar to the shape of a fork, which enables the user to simultaneously target opposing areas around the user's body, such as the front and the back of a shoulder.

Furthermore, the plurality of first rollers 8 is positioned adjacent to the distal arm end 5 of the first arm 2, as can be seen in FIGS. 4 and 5. Likewise, the plurality of second rollers 9 is positioned adjacent to the distal arm end 5 of the second arm 3. The positioning of the plurality of first rollers 8 and the plurality of second rollers 9 enables both to reach far body areas. In addition, each of the plurality of first rollers 8 is rotatably mounted onto the inner arm face 6 of the first arm 2. Each of the plurality of second rollers 9 is also rotatably mounted onto the inner arm face 6 of the second arm 3 as well. Thus, as the plurality of first rollers 8 and the plurality of second rollers 9 are pressed against the user's body, each of the plurality of first rollers 8 and each of the plurality of second rollers 9 can rotate and not hurt the user's body.

Both the plurality of first rollers 8 and the plurality of second rollers 9 are arranged to cover a wide area when pressed against the user's body. As can be seen in FIG. 4 through 6, the plurality of first rollers 8 and the plurality of second rollers 9 may each comprise a pair of terminal rollers 10 and a pair of intermediate rollers 11 to cover a larger body area. The pair of terminal rollers 10 is positioned in between the distal arm end 5 and the pair of intermediate rollers 11 to spread out the pair of terminal rollers 10 and the pair of intermediate rollers 11. The pair of terminal rollers 10 is also positioned opposite to each other across the inner arm face 6 to separate the pair of terminal rollers 10 from each other. Likewise, the pair of intermediate rollers 11 is positioned opposite to each other across the inner arm face 6 to separate the pair of intermediate rollers 11 from each other. In some embodiments, the pair of terminal rollers 10 is oriented away from each other so that each terminal roller is pressed against the user's body at an angle. Similarly, the pair of intermediate rollers 11 is oriented away from each other so that each intermediate roller is pressed against the user's body at an angle. Thus, any pressure applied onto the user's body using both the pair of terminal rollers 10 and the pair of intermediate rollers 11 does not hurt the user's body. In other embodiments, the plurality of first rollers 8 and the plurality of second rollers 9 can be rearranged to provide a different therapeutic benefit.

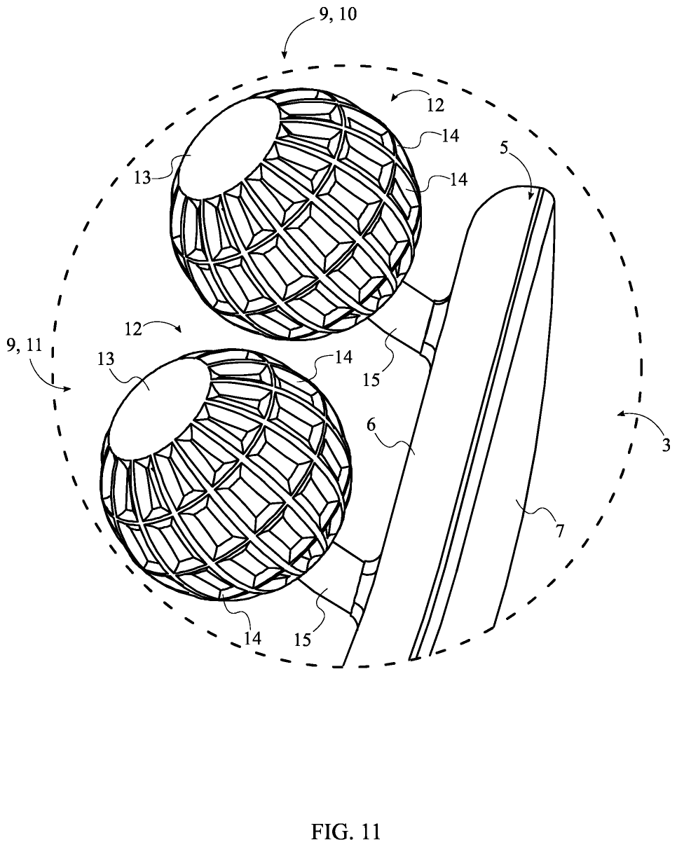

In addition, the plurality of first rollers 8 and the plurality of second rollers 9 are both designed to massage the user's body as each is pressed against the user's body. As can be seen in FIG. 11, each of the plurality of first rollers 8 and the plurality of second rollers 9 may further comprise a roller body 12, a plurality of recessions 14, and a roller stem 15. The roller body 12 is preferably the portion pressed against the user's body along with the plurality of recessions 14 to provide different therapeutic or cosmetic benefits. The roller stem 15 supports both the roller body 12 and the plurality of recessions 14. In addition, the roller body 12 may comprise an external body surface 13 that is pressed against the user's body. The roller stem 15 is rotatably connected to the inner arm face 6. In addition, the roller body 12 is torsionally connected to the roller stem 15. Thus, as the roller body 12 is pressed against the user's body and rotates, the roller stem 15 also rotates with the roller body 12 and does not transfer torque onto the corresponding arm. The roller body 12 is also positioned offset from the inner arm face 6 so that both the roller body 12 and the roller stem 15 are free to rotate. Further, each of the plurality of recessions 14 is integrated into the external body surface 13. The plurality of recessions 14 is also distributed about the external body surface 13. Thus, as the roller body 12 is pressed against the user's body and rotates, different recessions of the plurality of recessions 14 are in contact with the user's body. In some embodiments, the roller body 12 is preferably a spherical body to facilitate the rotation of the roller body 12 against the user's body. In other embodiments, the roller body 12 can be shaped differently to provide other therapeutic or cosmetic benefits.

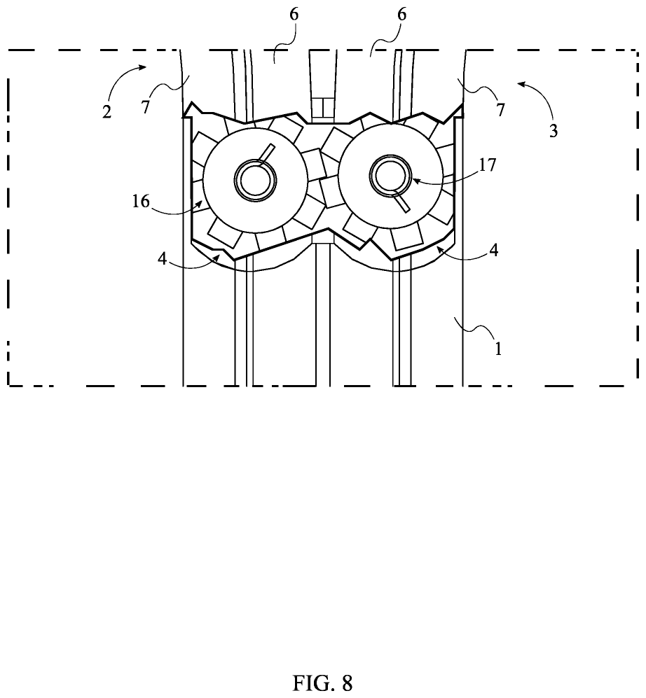

To enable the first arm 2 to mirror the positioning of the second arm 3 and vice versa, the present invention may further comprise a geared continuous hinge 16. As can be seen in FIG. 8, the geared continuous hinge 16 enables both the first arm 2 and the second arm 3 to mirror each other as both the first arm 2 and the second arm 3 pivot about the corresponding proximal arm end 4. Further, the proximal arm end 4 of the first arm 2 is operatively coupled to the proximal arm end 4 of the second arm 3 by the geared continuous hinge 16, wherein the geared continuous hinge 16 is used to mirror a movement by the first arm 2 with a movement by the second arm 3 and is used to mirror a movement by the second arm 3 with a movement by the first arm 2. For example, the geared continuous hinge 16 may include multiple gears interconnected in between the proximal arm end 4 of the first arm 2 and the proximal arm end 4 of the second arm 3 so that the pivoting of one proximal arm end 4 causes the other proximal arm end 4 to pivot in the opposite direction. In other embodiments, the geared continuous hinge 16 may be replaced with a different mechanism that facilitates the mirroring of the first arm 2 and the second arm 3.

In addition to the geared continuous hinge 16, the present invention may further comprise a spring-loaded mechanism 17 that enables the first arm 2 and the second arm 3 to return to the starting position after the first arm 2 and the second arm 3 have been pressed against the user's body. As can be seen in FIG. 8, the proximal arm end 4 of the first arm 2 is operatively coupled to the proximal arm end 4 of the second arm 3 by the spring-loaded mechanism 17, wherein the spring-loaded mechanism 17 is used to recoil the first arm 2 to a first rest position and is used to recoil the second arm 3 to a second rest position, and wherein the first rest position and the second rest position is positioned offset to each other. For example, the spring-loaded mechanism 17 may include a hinged spring integrated into the proximal arm end 4 of the first arm 2 and another hinged spring integrated into the proximal arm end 4 of the second arm 3. Thus, the first arm 2 and the second arm 3 can return to the corresponding starting rest position after the present invention has been used. In other embodiments, the spring-loaded mechanism 17 can be replaced with a different mechanism that enables the recoiling of the first arm 2 and the second arm 3.

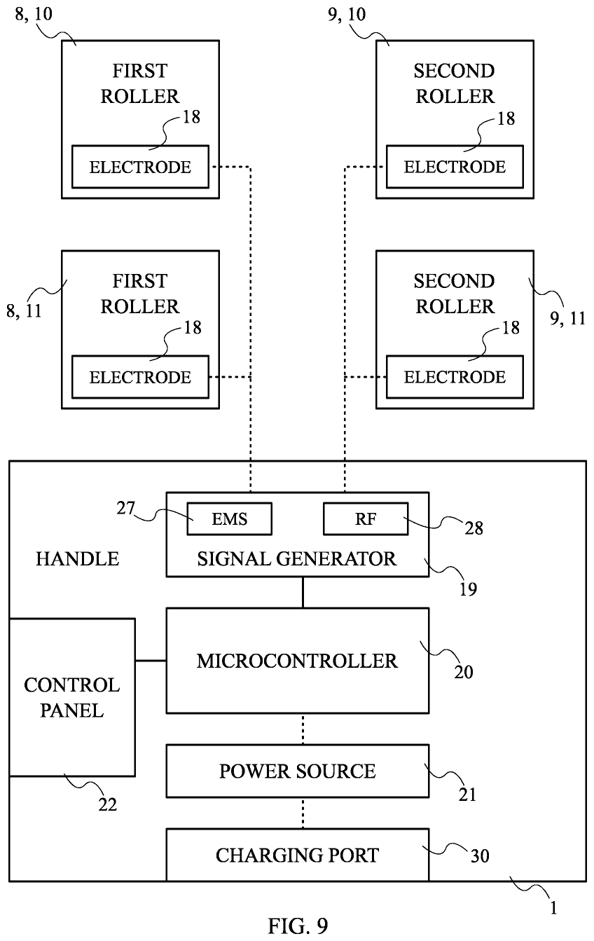

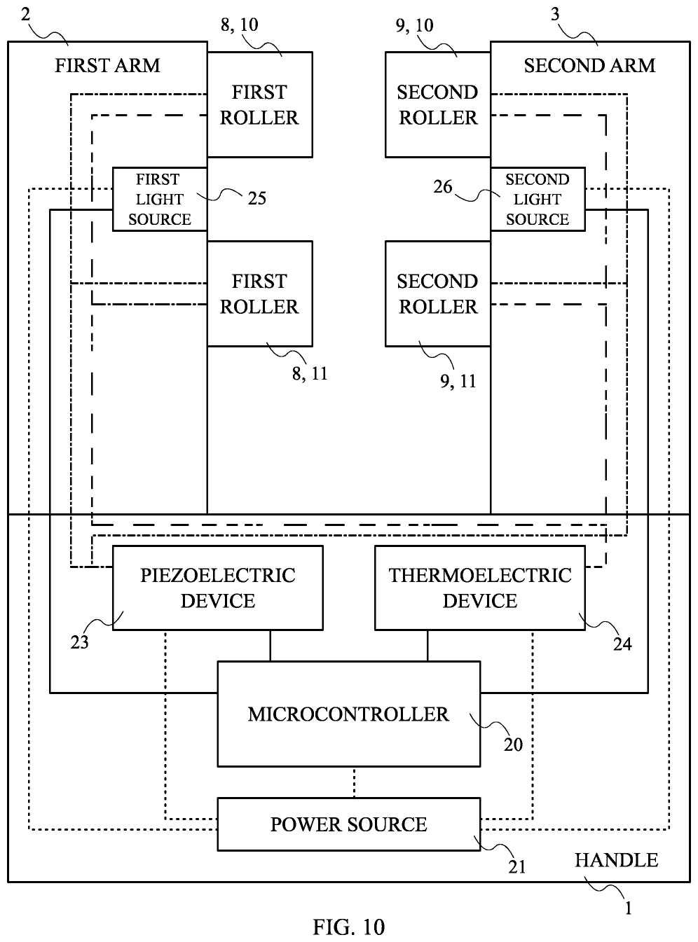

In some embodiments, the present invention is preferably equipped with various cosmetic and therapeutic technologies to increase the effectiveness of the present invention. As can be seen in FIGS. 9 and 10, the present invention may further comprise a plurality of electrodes 18, a signal generator 19, a microcontroller 20, and a power source 21. The plurality of electrodes 18 serves to apply an electrical current generated by the signal generator 19. The microcontroller 20 controls the operation of the signal generator 19 by controlling the power transmitted to the signal generator 19 from the power source 21. In addition, each of the plurality of first rollers 8 and each of the plurality of second rollers 9 is made of an electrically conductive material to transmit the electrical current from the signal generator 19. The signal generator 19, the microcontroller 20, and the power source 21 are mounted within the handle 1 to house each component from the environment. Each of the plurality of electrodes 18 is integrated into a corresponding roller of the plurality of first rollers 8 and the plurality of second rollers 9 to transmit the electrical current from the signal generator 19 to the corresponding roller. Further, the microcontroller 20 is electronically connected to the signal generator 19 to control the operation of the signal generator 19. The signal generator 19 is electrically connected to the plurality of electrodes 18 to transmit electrical signals. Finally, the power source 21 is electrically connected to the microcontroller 20 and the signal generator 19 to provide the power for the operation of the microcontroller 20 and the signal generator 19. The power source 21 is preferably a rechargeable battery that can be recharged using a charging port 30 that is laterally integrated into the handle 1. In other embodiments, the power source 21 may be one or more replaceable batteries.

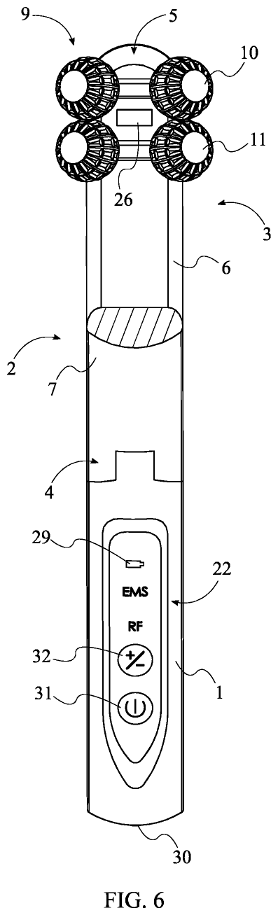

Furthermore, the present invention may further comprise a control panel 22 to enable the user to configure the operation of the signal generator 19 through the microcontroller 20. As can be seen in FIGS. 6, 9, and 10, the control panel 22 is laterally integrated into the handle 1 so that the control panel 22 is accessible to the user. In addition, the control panel 22 is electronically connected to the control panel 22 to transmit the user's inputs. In some embodiments, the control panel 22 can also provide information regarding the current operation of the present invention. The control panel 22 can include at least one indicator 29 showing the power levels of the power source 21, the selected mode of operation of the signal generator 19, etc. Further, the at least one indicator 29 can alert the user of any errors or other operational issues, such as low battery. The control panel 22 can also provide multiple buttons with various functions to enable the configuration of the present invention. The control panel 22 can further include a power button 31 and an intensity button 32 that enables the user power on or off the present invention and control the intensity of the signal generator 19, respectively. In other embodiments, the control panel 22 can provide other features to facilitate the control of the present invention by the user.

As previously discussed, the present invention is able to provide different cosmetic and therapeutic technologies to increase the effectiveness of the healing and rejuvenating benefits of the present invention. As can be seen in FIG. 10, the present invention may further comprise a piezoelectric device 23 designed to generate vibrations for massaging purposes. The piezoelectric device 23 is mounted within the handle 1 to protect the piezoelectric device 23 from the environment. The piezoelectric device 23 is also in vibrational communication with the plurality of first rollers 8 to transmit the vibrations generated by the piezoelectric device 23 to the plurality of first rollers 8. Likewise, the piezoelectric device 23 is in vibrational communication with the plurality of second rollers 9 to transmit the vibrations generated by the piezoelectric device 23 to the plurality of second rollers 9. Furthermore, the microcontroller 20 is electronically connected to the piezoelectric device 23 so that the user can configure the operation of the piezoelectric device 23. The power source 21 is also electrically connected to the piezoelectric device 23 to provide the power necessary for the operation of the piezoelectric device 23. In other embodiments, the present invention may utilize different devices to generate vibrations to massage the user's body.

In addition to the piezoelectric device 23, the present invention may further comprise a thermoelectric device 24 to provide hot and cold therapy, as can be seen in FIG. 10. Like the piezoelectric device 23, the thermoelectric device 24 is mounted within the handle 1 to protect the piezoelectric device 23 from the environment and any possible damage. The thermoelectric device 24 is in thermal communication with the plurality of first rollers 8 to transmit the thermal energy generated by the thermoelectric device 24 to the plurality of first rollers 8. Likewise, the thermoelectric device 24 is in thermal communication with the plurality of second rollers 9 to transmit the thermal energy generated by the thermoelectric device 24 to the plurality of second rollers 9. Furthermore, the microcontroller 20 is electronically connected to the thermoelectric device 24 so that the user can configure the operation of the thermoelectric device 24. For example, the user can select heat to be applied to the target area by configuring the thermoelectric device 24 to generate heat to warm up the rollers of the plurality of first rollers 8 and the plurality of second rollers 9. Alternatively, the user can select cold to be applied to the target area by configuring the thermoelectric device 24 to cool down the rollers of the plurality of first rollers 8 and the plurality of second rollers 9. The power source 21 is also electrically connected to the thermoelectric device 24 to provide the power necessary for the operation of the thermoelectric device 24. In other embodiments, the present invention may utilize other devices to generate different thermal energy to treat the user's body.

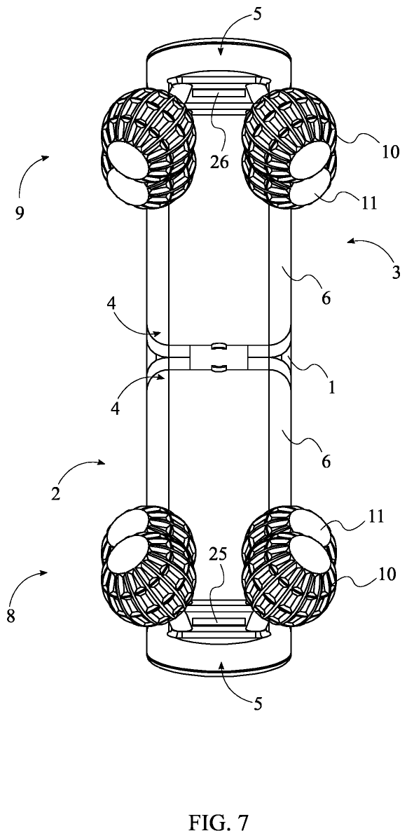

Furthermore, the present invention may further comprise a first light source 25 and a second light source 26 to provide light therapy to the desired target body areas. As can be seen in FIGS. 6 and 7, the first light source 25 and the second light source 26 can be configured to emit light in different frequencies, such as in the infrared light spectrum. In addition, different types of light sources can be utilized for the first light source 25 and the second light source 26, such as LED lights. The first light source 25 and the second light source 26 are positioned on the first arm 2 and the second arm 3, respectively, to fully reach the desired target body areas. The first light source 25 is integrated into the inner arm face 6 of the first arm 2 so that the first light source 25 is oriented towards the user's body. The first light source 25 is also positioned amongst the plurality of first rollers 8 so that the target body area is exposed to the first light source 25. Further, the microcontroller 20 is electronically connected to the first light source 25 so that the user can configure the operation of the first light source 25. The power source 21 is also electrically connected to the first light source 25 to provide the power necessary for the operation of first light source 25.

Similar to the first light source 25, the second light source 26 is integrated into the inner arm face 6 of the second arm 3 so that the second light source 26 is oriented towards the user's body. As can be seen in FIGS. 6 and 7, the second light source 26 is also positioned amongst the plurality of second rollers 9 so that the target body area is exposed to the second light source 26. Further, the microcontroller 20 is electronically connected to the second light source 26 so that the user can configure the operation of the second light source 26. The power source 21 is also electrically connected to the second light source 26 to provide the power necessary for the operation of second light source 26. In other embodiments, multiple light sources can be provided on either the first arm 2 or the second arm 3 to target a large body area.

In addition to light therapy and hot and cold therapy, the present invention is able to provide different stimulation therapies using the signal generator 19, as can be seen in FIG. 9. In one embodiment, the signal generator 19 may be an Electrical Muscle Stimulation (EMS) signal generator 27 that can be used for physical therapy and various fitness techniques. EMS therapy enables users to treat muscles as well as other body areas. The user can selectively activate the EMS signal generator 27 to target the desired body area. In another embodiment, the signal generator 19 may be a Radio Frequency (RF) signal generator 28 that can be used to treat the user's skin. RF therapy enables the user to target the dermis to tighten the surrounding skin areas. Like the EMS signal generator 27, the RF signal generator 28 can be selectively activated to treat the desired body area. Furthermore, the present invention can include multiple types of signal generators which can be selectively activated using the control panel 22. The multiple signal generators can be a combination of the EMS signal generator 27, the RF signal generator 28, etc. and each can be individually or simultaneously controlled using the microcontroller 20. In other embodiments, the present invention can utilize different types of signal generators to provide different types of therapy.

Although the invention has been explained in relation to its preferred embodiment, it is to be understood that many other possible modifications and variations can be made without departing from the spirit and scope of the invention.

What is claimed is:

1. A rejuvenating beauty care tool for skin and body comprising:
a handle;
a first arm;
a second arm;
a plurality of first rollers;
a plurality of second rollers;
the first arm and the second arm each comprising a proximal arm end, a distal arm end, an inner arm face, and an outer arm face;
the proximal arm end being hingedly and terminally connected to the handle;
the inner arm face of the first arm and the inner arm face of the second arm being oriented towards each other;
the outer arm face of the first arm and the outer arm face of the second arm being oriented away from each other;
the plurality of first rollers being positioned adjacent to the distal arm end of the first arm;
the plurality of second rollers being positioned adjacent to the distal arm end of the second arm;
each of the plurality of first rollers being rotatably mounted onto the inner arm face of the first arm; and,
each of the plurality of second rollers being rotatably mounted onto the inner arm face of the second arm.

2. The rejuvenating beauty care tool for skin and body as claimed in claim 1 comprising:
the plurality of first rollers and the plurality of second rollers each comprising a pair of terminal rollers and a pair of intermediate rollers;
the pair of terminal rollers being positioned in between the distal arm end and the pair of intermediate rollers;
the pair of terminal rollers being positioned opposite to each other across the inner arm face; and,
the pair of intermediate rollers being positioned opposite to each other across the inner arm face.

3. The rejuvenating beauty care tool for skin and body as claimed in claim 2 comprising:
the pair of terminal rollers being oriented away from each other.

4. The rejuvenating beauty care tool for skin and body as claimed in claim 2 comprising:
the pair of intermediate rollers being oriented away from each other.

5. The rejuvenating beauty care tool for skin and body as claimed in claim 1 comprising:
each of the plurality of first rollers and each of the plurality of second rollers each comprising a roller body, a plurality of recessions, and a roller stem;
the roller body comprising an external body surface;
the roller stem being rotatably connected to the inner arm face;
the roller body being torsionally connected to the roller stem;
the roller body being positioned offset from the inner arm face;
each of the plurality of recessions being integrated into the external body surface; and,
the plurality of recessions being distributed about the external body surface.

6. The rejuvenating beauty care tool for skin and body as claimed in claim 5, wherein the roller body is a spherical body.

7. The rejuvenating beauty care tool for skin and body as claimed in claim 1 comprising:
a geared continuous hinge; and,
the proximal arm end of the first arm being operatively coupled to the proximal arm end of the second arm by the geared continuous hinge, wherein the geared continuous hinge is used to mirror a movement by the first arm with a movement by the second arm and is used to mirror a movement by the second arm with a movement by the first arm.

8. The rejuvenating beauty care tool for skin and body as claimed in claim 1 comprising:
a spring-loaded mechanism; and,
the proximal arm end of the first arm being operatively coupled to the proximal arm end of the second arm by the spring-loaded mechanism, wherein the spring-loaded mechanism is used to recoil the first arm to a first rest position and is used to recoil the second arm to a second rest position, and wherein the first rest position and the second rest position is positioned offset to each other.

9. The rejuvenating beauty care tool for skin and body as claimed in claim 1 comprising:
a plurality of electrodes;
a signal generator;
a microcontroller;
a power source;
each of the plurality of first rollers and each of the plurality of second rollers being made of an electrically conductive material;
the signal generator, the microcontroller, and the power source being mounted within the handle;
each of the plurality of electrodes being integrated into a corresponding roller of the plurality of first rollers and the plurality of second rollers;
the microcontroller being electronically connected to the signal generator;
the signal generator being electrically connected to the plurality of electrodes; and,
the power source being electrically connected to the microcontroller and the signal generator.

10. The rejuvenating beauty care tool for skin and body as claimed in claim 9 comprising:
a control panel;
the control panel being laterally integrated into the handle; and,
the microcontroller being electronically connected to the control panel.

11. The rejuvenating beauty care tool for skin and body as claimed in claim 9 comprising:
a piezoelectric device;
the piezoelectric device being mounted within the handle;
the piezoelectric device being in vibrational communication with the plurality of first rollers;
the piezoelectric device being in vibrational communication with the plurality of second rollers;
the microcontroller being electronically connected to the piezoelectric device; and,
the power source being electrically connected to the piezoelectric device.

12. The rejuvenating beauty care tool for skin and body as claimed in claim 9 comprising:
a thermoelectric device;
the thermoelectric device being mounted within the handle;
the thermoelectric device being in thermal communication with the plurality of first rollers;
the thermoelectric device being in thermal communication with the plurality of second rollers;
the microcontroller being electronically connected to the thermoelectric device; and,
the power source being electrically connected to the thermoelectric device.

13. The rejuvenating beauty care tool for skin and body as claimed in claim 9 comprising:
a first light source;
the first light source being integrated into the inner arm face of the first arm;
the first light source being positioned amongst the plurality of first rollers;
the microcontroller being electronically connected to the first light source; and,
the power source being electrically connected to the first light source.

14. The rejuvenating beauty care tool for skin and body as claimed in claim 9 comprising:
a second light source;
the second light source being integrated into the inner arm face of the second arm;
the second light source being positioned amongst the plurality of second rollers;
the microcontroller being electronically connected to the second light source; and,
the power source being electrically connected to the second light source.

15. The rejuvenating beauty care tool for skin and body as claimed in claim 9, wherein the signal generator is an electrical muscle stimulation (EMS) signal generator.

16. The rejuvenating beauty care tool for skin and body as claimed in claim 9, wherein the signal generator is a radio frequency (RF) signal generator.
